(19) United States
(12) Patent Application Publication
(10) Pub. No.: US 2025/0009429 A1
(43) Pub. Date: Jan. 9, 2025

(54) MEDICAL OPTICAL FIBER WITH INTRA-PROCEDURE RENEWABLE TIP

(71) Applicant: Boston Scientific Scimed, Inc., Maple Grove, MN (US)

(72) Inventors: Jian Zhang, Santa Clara, CA (US); Alex Lemieux, Southborough, MA (US); Steven Yihlih Peng, Fremont, CA (US); Caroline Brial, Boston, MA (US); Michael O'Brien, Newton, MA (US)

(73) Assignee: Bosto Scientific Scimed, Inc., Maple Grove, MN (US)

(21) Appl. No.: 18/764,730

(22) Filed: Jul. 5, 2024

Related U.S. Application Data
(60) Provisional application No. 63/512,266, filed on Jul. 6, 2023.

(51) Int. Cl. A61B 18/26; A61B 18/22

(52) U.S. Cl. CPC A61B 18/26; A61B 2018/2205

(57) ABSTRACT

The present disclosure provides a medical optical fiber comprising a renewable distal end. The renewable distal end comprising several detachable segments where the detachable segments can be selectively and/or automatically detached to renew the distal most tip of the optical fiber.

### CROSS-REFERENCE TO RELATED APPLICATIONS

This application claims the benefit of U.S. Provisional Patent Application Ser. No. 63/512,266 filed on Jul. 6, 2023, the disclosure of which is incorporated herein by reference.

### TECHNICAL FIELD

The present disclosure generally relates to medical optical fibers. Particularly, but not exclusively, the present disclosure relates to renewing the tip on the distal end of the medical optical fiber intra-procedure and specifically without removing the fiber from an endoscope.

### BACKGROUND

Medical optical fibers typically include an optical fiber and a jacket surrounding the optical fiber. Optical fibers may comprise an innermost optical core and optionally a cladding layer surrounding the optical core. Some optical fibers can optionally include a mechanical support layer surrounding the cladding layer. Additionally, medical optical fibers often include jackets (or protective layers) and many have portions of their jackets stripped at a distal end (e.g., similar to electrical wires) leaving a bare optical fiber at the distal end. Medical optical fiber diameters are in the range of a few tens of microns to a few hundreds of microns.

A common use for medical optical fibers is in ureteroscopic laser lithotripsy (URSL). During a URSL procedure, a flexible endoscope is inserted through a patient's urinary channel to access the kidney region. A small core fiber (sub 300 μm) is inserted through a working channel of the flexible endoscope and used to treat (e.g., fragment, ablate, etc.) a stone in the kidney region. It is to be appreciated that during such a procedure, laser energy is emitted from the distal end of the fiber, which can cause degradation of the distal tip before the procedure is completed.

The Holmium-doped yttrium aluminum garnet (Ho:YAG) laser has been the favored lithotripter for the treatment of urinary calculus since shortly after its introduction in the 1990s because it can fragment all calculus compositions and produces less calculus migration (retropulsion) during treatment than the short-pulsed lasers. As more and more small core fibers become available for high-power laser application (>20 W), fiber distal tip degradation during URSL has become a more critical issue as the small core fibers have lower mechanical strength and higher laser energy intensity versus larger core fibers.

### BRIEF SUMMARY

The present disclosure provides a medical optical fiber comprising an optical fiber disposed along a longitudinal axis. The medical optical fiber further includes an optical fiber distal tip configured to be reconditioned intra-procedure without removing the tip from an endoscope. Further, some embodiments provide an endoscope configured to recondition the distal tip of the optical fiber.

Embodiments of the disclosure can be implemented as a medical optical fiber. The medical optical fiber can comprise a non-renewable portion; and a renewable portion coupled to a distal end of the non-renewable portion, the renewable portion comprising a plurality of detachable segments.

In further embodiments of the medical optical fiber, each of the plurality of detachable segments comprises an optically transparent material having a generally cylindrical shape.

In further embodiments of the medical optical fiber, each of the plurality of detachable segments has a length between 0.2 millimeters (mm) and 6.0 mm.

In further embodiments of the medical optical fiber, each of the plurality of detachable segments can comprise a ferrous material, the ferrous material configured to interact with an electromagnetic field to selectively decouple one of the plurality of detachable segments.

In further embodiments of the medical optical fiber, the medical optical fiber configured to be inserted through a working channel of an endoscope, the endoscope comprising a catheter having one or more electromagnets disposed at a distal end, the one or more electromagnets configured to generate the electromagnetic field.

In further embodiments of the medical optical fiber, the plurality of detachable segments are coupled to each other via an adhesive material.

In further embodiments of the medical optical fiber, the adhesive material is configured to melt, dissolve, or substantially weaken when exposed to specific wavelengths of light, when exposed to a select combination of laser pulse frequency and/or power, upon reaching a specific temperature, or upon exposure to a quantity of transmitted laser energy.

In further embodiments of the medical optical fiber, the adhesive material between a first one of the plurality of detachable segments and a second one of the plurality of detachable segments is configured to melt when exposed to a first quantity of laser energy, wherein the adhesive material between the second one of the plurality of detachable segments and a third one of the plurality of detachable segments is configured to melt when exposed to a second quantity of laser energy, wherein the second one of the plurality of detachable segments is proximal to the first one of the plurality of detachable segments, wherein the third one of the plurality of detachable segments is proximal to the second one of the plurality of detachable segments, and wherein the second quantity of laser energy is higher than the first quantity of laser energy.

In further embodiments of the medical optical fiber, each of the plurality of detachable segments comprises a machined end surface, wherein the machined end surface of a first one of the plurality of detachable segments is configured to attract via intermolecular attraction the machined end surface of a second one of the plurality of detachable segments when the end surfaces are positioned proximate to each other.

Embodiments of the disclosure can be implemented as an endoscope system configured for use with a renewable optical fiber. The system can comprise an endoscope having a working channel; and a renewable optical fiber configured to be inserted through the working channel, the renewable optical fiber comprising a non-renewable portion; and a renewable portion at a distal end of the non-renewable portion, the renewable portion comprising a plurality of detachable segments.

In further embodiments of the endoscope system, the endoscope can comprise a catheter, the working channel extending from a proximal end to a distal end of the catheter, the distal end of the catheter comprising a cleaving apparatus configured to cleave the plurality of detachable segments.

In further embodiments of the endoscope system, the endoscope can comprise a handle, the handle comprising an actuator arranged to actuate the cleaving apparatus.

In further embodiments of the endoscope system, each of the plurality of detachable segments comprises an optically transparent material having a generally cylindrical shape.

In further embodiments of the endoscope system, each of the plurality of detachable segments have a length between 0.2 millimeters (mm) and 6.0 mm.

In further embodiments of the endoscope system, the endoscope can comprise a catheter, the working channel extending from a proximal end to a distal end of the catheter, the distal end of the catheter comprising one or more electromagnets, wherein each of the plurality of detachable segments comprise a ferrous material, the ferrous material configured to interact with an electromagnetic field generated by the one or more electromagnets to selectively decouple a one of the plurality of detachable segments.

Embodiments of the disclosure can be implemented as lithotripsy method comprising providing a medical optical fiber having a plurality of detachable segments coupled to a distal end of the medical optical fiber; inserting the medical optical fiber through a working channel of an endoscope; irradiating a target with laser energy via the medical optical fiber; and renewing the distal end of the medical optical fiber by detaching a most distal one of the plurality of detachable segments.

In further embodiments of the lithotripsy method, each of the plurality of detachable segments comprises an optically transparent material having a generally cylindrical shape.

In further embodiments of the lithotripsy method, each of the plurality of detachable segments has a length between 0.2 millimeters (mm) and 6.0 mm.

In further embodiments of the lithotripsy method, each of the plurality of detachable segments can comprise a ferrous material, the ferrous material configured to interact with an electromagnetic field to selectively decouple one of the plurality of detachable segments.

In further embodiments of the lithotripsy method, the endoscope can comprise a catheter having one or more electromagnets disposed at a distal end, the one or more electromagnets configured to generate the electromagnetic field.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A and FIG. 1B illustrate an endoscope system in accordance with at least one embodiment of the present disclosure.

FIG. 2 illustrates a medical optical fiber with a renewable distal tip in accordance with at least one embodiment of the present disclosure.

FIG. 3 illustrates another medical optical fiber and a distal end of an endoscope configured to renew the distal top of the optical fiber in accordance with at least one embodiment of the present disclosure.

FIG. 4 illustrates another medical optical fiber with a renewable distal tip in accordance with at least one embodiment of the present disclosure.

FIG. 5 illustrates yet another medical optical fiber with a renewable distal tip in accordance with at least one embodiment of the present disclosure.

FIG. 6 illustrates a method in accordance with at least one embodiment of the present disclosure.

### DETAILED DESCRIPTION

As noted, the present disclosure provides apparatuses and methods for renewing lithotripsy laser fibers that can mitigate problems inherent in ureteroscope laser lithotripsy (URSL) procedures that cause the fiber distal-tip to degrade. The present disclosure provides several examples or embodiments of such a “self-renewing” optical fiber. It is to be appreciated that although these examples are described as separate embodiments features from one example could be combined with features from another example without departing from the spirit of the disclosure.

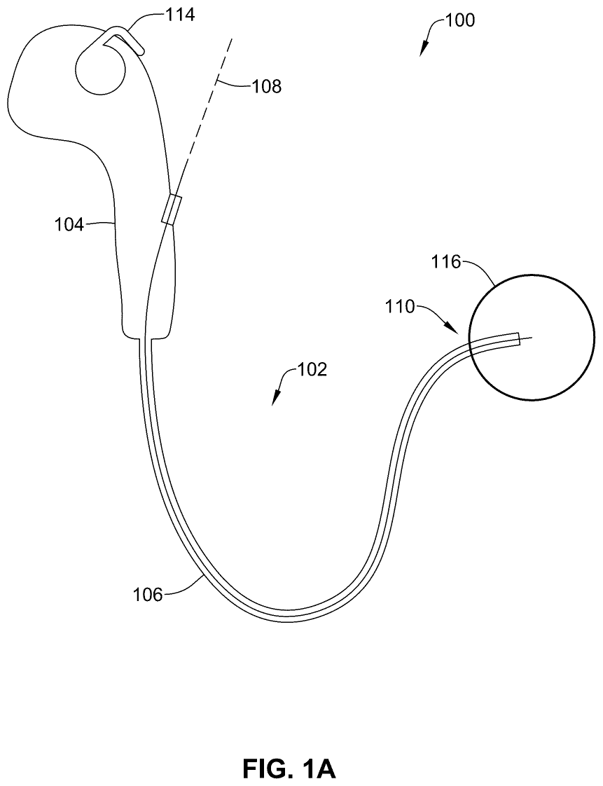

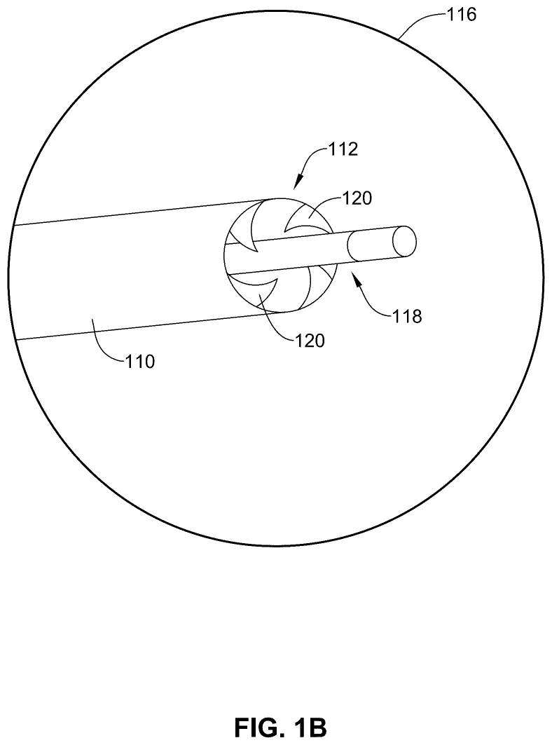

FIG. 1A and FIG. 1B illustrates an endoscope system 100 with an integrated cleaving apparatus configured to renew the distal end of an optical fiber. As depicted, the endoscope system 100 includes an endoscope 102 having a handle 104 and catheter 106. The catheter 106 has at least one working channel in which an optical fiber 108 can be inserted. At the distal end of the catheter 106 is a cleaving apparatus 112 that can be actuated by actuator 114, which is disposed on handle 104, to renew the distal end (118) of optical fiber 108. This is depicted in greater detail by enlarged view 116 shown in FIG. 1B.

As can be seen from enlarged view 116 in FIG. 1B, the distal end 110 of catheter 106 includes cleaving apparatus 112. The distal end of optical fiber 118 can be extended out from the distal end 110 and the cleaving apparatus 112 can be actuated by actuator 114 to cleave off the distal end of optical fiber 118 to expose a fresh end of optical fiber 108.

In some embodiments, the cleaving apparatus 112 can comprise teeth 120 having a razor edge arranged to cut or cleave the distal end of optical fiber 118. The teeth can be arranged in a circular fashion and configured to collapse or rotate inward to compress the opening of the working channel in which the optical fiber 108 is inserted so that the teeth 120 cut or cleave off the distal end of optical fiber 118. It is to be appreciated that the optical fiber 108 can often comprise an inner optical fiber core and one or more covering layers (e.g., cladding, or the like). With some embodiments, the teeth 120 can be configured to strip (e.g., the cladding, etc.) from a portion of the distal end of optical fiber 118 to expose a fresh portion of the inner optical fiber core and then to cleave the exposed optical fiber core to renew the distal end of optical fiber 118.

Once the distal end of optical fiber 118 is renewed, the optical fiber 108 can be extended further through the working channel of catheter 106 to expose an operating length of the newly renewed distal end of optical fiber 118.

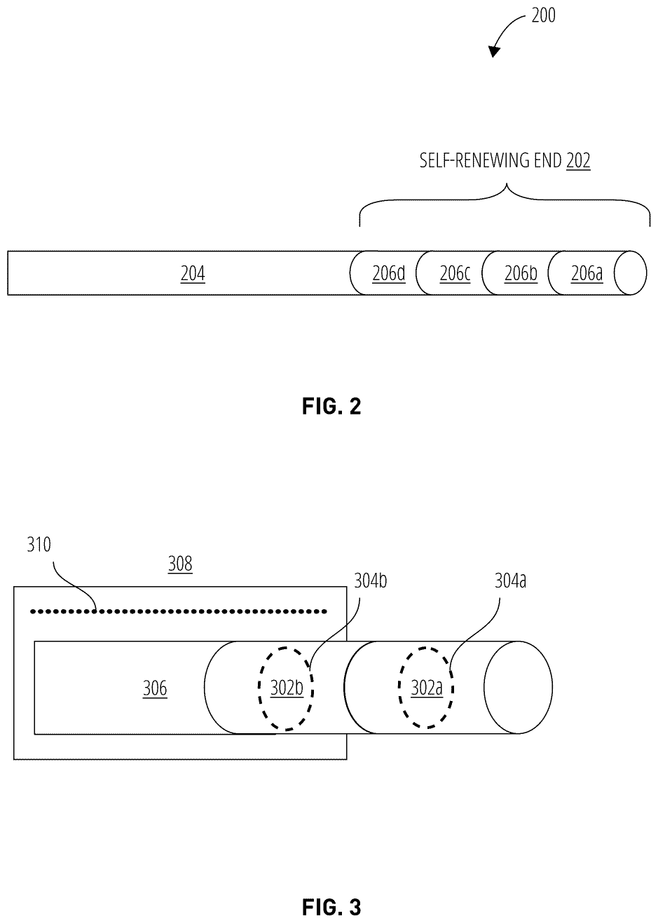

FIG. 2 illustrates an optical fiber 200, according to various embodiments of the present disclosure. The optical fiber 200 includes a self-renewing end 202 disposed at a distal end of a non-renewable portion 204. The non-renewable portion 204 can comprise an optical fiber core and one or more coverings (e.g., cladding, shielding, protective layer, etc.). The self-renewing end 202 comprises several (e.g., one or more) detachable segments or “bullets.” As depicted, self-renewing end 202 includes detachable segments 206a, 206b, 206c, and 206d. It is noted that more or less than four (4) detachable segments couple be provided. However, hour (4) segments are used in this example for purposes of clarity of presentation. Each one of the detachable segments 206a to 206d comprises a rod-shaped optically transparent section ranging from a 0.2 millimeters (mm) to 6.0 mm in length. Each one of the detachable segments 206a to 206d can detach from the next most proximal segment (e.g., after exposure to a specific quantity of laser energy, after a quantifiable amount of degradation). With some embodiments, the segment can detach from the next proximal segment in an automatic manner. In some embodiments, the segment can detach from the next proximal segment in a controller or user actuated manner.

With some embodiments, the interface between each of the detachable segments 206a, 206b, 206c, and 206d can be adhesive, optical diffused, or electro-magnetically joined. It is to be appreciated that the detached segments (e.g., detachable segment 206a, or the like) can be small enough (e.g., less than 1.0 millimeters (mm), less than 0.5 mm, or the like) that a patient may pass the detached segments (e.g., through the urinary system, or the like) in a natural manner.

As such, optical fiber 200 provides an optical fiber that be renewed intra-procedure without removing the optical fiber 200 from the endoscope. As such, the distal end of the optical fiber 200 can be maintained with a relatively high transmission rate to the laser beam to ensure an effective laser treatment during the whole procedure.

FIG. 3 illustrates an embodiment of detachable segments 302a and 302b, which can be provided as detachable segments 206a to 206d of optical fiber 200 of FIG. 2. In general, detachable segments 302a and 302b are coupled to the end of an optical fiber via electromagnetic fields. Each of the detachable segments 302a and 302b include a ferrous wire 304a and 304b, respectively, which can be acted upon by an electromagnetic field to retain the segments in position until a user chooses to detach an individual segment. For example, electromagnets 310 can be positioned near a distal end of a catheter of an endoscope 308 (e.g., 110 of catheter 106, or the like) and activated to load several detachable segments (e.g., 302a, 302b, etc.) onto a distal end of a renewable optical fiber 306. As the distal end of the optical fiber on which the detachable segments 302a and 302b are loaded is advanced out of the distal end of the endoscope, the electromagnetic field created by the electromagnets 310 will weaken and the most distal segment (e.g., detachable segment 302a, or the like) will drop off renewing the end of the optical fiber.

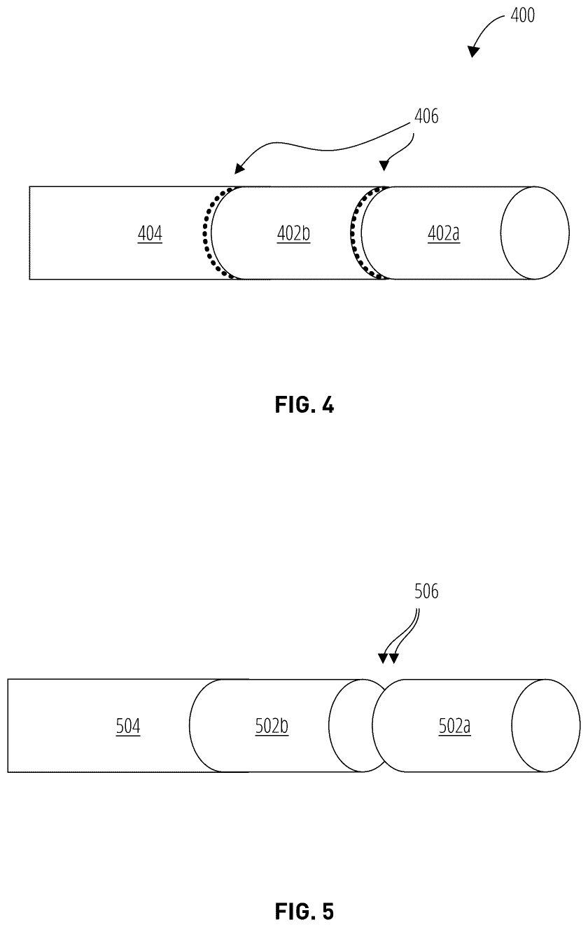

FIG. 4 illustrates an embodiment of detachable segments 402a and 402b, which can be provided as detachable segments 206a to 206d of optical fiber 200 of FIG. 2. In general, detachable segments 402a and 402b are coupled to the end of an optical fiber via an adhesive 406. Detachable segments 402a and 402b can be coupled to renewable optical fiber 404 via adhesive 406 where the adhesive is configured to weaken, melt, or dissolve, based on select conditions. For example, adhesive 406 can be configured to dissolve when exposed to specific wavelengths of light, when exposed to a select frequency and/or power combination of laser energy, upon reaching a specific temperature, or upon exposure to a quantity of transmitted energy.

For example, adhesive 406 could be configured to melt at a specific temperature such that once a particular segment (e.g., detachable segment 402a, or the like) is exposed to the operating environment and heats to a particular temperature that might indicate degradation of the distal-most tip of the segment, the adhesive 406 coupling that particular segment to the next proximal segment will melt allowing the segment to fall off and the optical fiber 400 to be advanced such that the remaining distal segment is exposed to a proper operating length.

As another example, adhesive 406 coupling detachable segment 402a and detachable segment 402b could be configured to melt after exposure to a specified quantity of laser energy while adhesive 406 coupling detachable segment 402b and renewable optical fiber 404 could be configured to melt after exposure to a higher specified quantity of laser energy. In such an example, detachable segment 402a can be configured to fall off (e.g., due to adhesive 406 between detachable segments 402a and 402b dissolving) prior to detachable segment 402b falling off.

FIG. 5 illustrates an embodiment of detachable segments 502a and 502b, which can be provided as detachable segments 206a to 206d of optical fiber 200 of FIG. 2. In general, detachable segments 502a and 502b are coupled to the end of an optical fiber via intermolecular attraction. Detachable segments 502a and 502b can be coupled to renewable optical fiber 504 based on machined faces 506 being sufficiently flat such that when machined faces 506 are brought in proximity to each other, an intermolecular attraction adheres the detachable segment 502a to the detachable segment 502b.

During operation, ones of detachable segments 502a and/or 502b can be removed by tapping the segment to be removed with a tool inserted through another working channel of the endoscope.

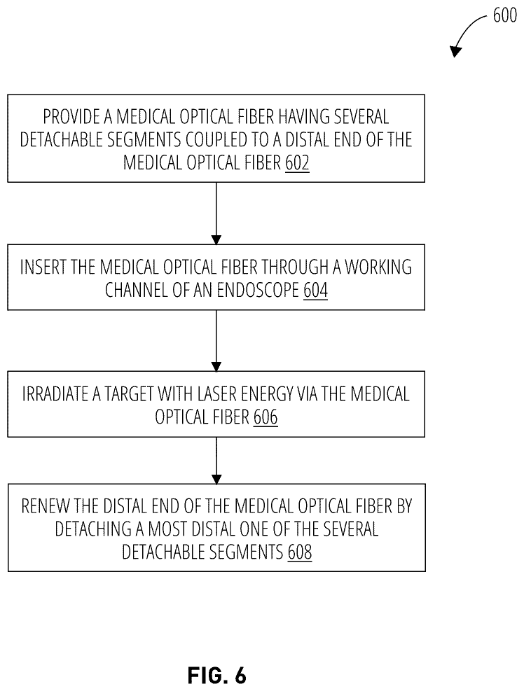

FIG. 6 illustrates method 600 of renewing a distal end of a medical optical fiber. Method 600 can begin at block 602. At block 602 “provide a medical optical fiber having several detachable segments coupled to a distal end of the medical optical fiber” a medical optical fiber having several detachable segments coupled to a distal end of the medical optical fiber can be provided. For example, optical fiber 200 having self-renewing end 202 detachable segments 206a to 206d coupled to non-renewable portion 204 can be provided.

Continuing to block 604 “insert the medical optical fiber through a working channel of an endoscope” the medical optical fiber can be inserted through a working channel of an endoscope. For example, optical fiber 200 can be inserted through a working channel of endoscope 102 like optical fiber 108 as depicted in FIG. 1A.

Continuing to block 606 “irradiate a target with laser energy via the medical optical fiber” a target can be irradiated with laser energy via the medical optical fiber. For example, optical fiber 200 can be coupled to a laser source (not shown) where the laser source is arranged to generate laser energy and direct the laser energy to a target through the medical optical fiber. For example, optical fiber 200 can be coupled at a proximal end to a laser source and the distal end (e.g., self-renewing end 202) positioned proximate to a target (e.g., stone, tissue, or the like) through endoscope 102. The target can be irradiated with laser energy via the optical fiber 200.

Continuing to block 608 “renew the distal end of the medical optical fiber by detaching a most distal one of the several detachable segments” the distal end of the medical optical fiber can be renewed by detaching one of the detachable segments. For example, one of the detachable segments 206a to 206d can be detached by renewing the self-renewing end 202. It is to be appreciated that as the target is irradiated with laser energy (e.g., at block 606, or the like) the distal end of the medical optical fiber (e.g., self-renewing end 202, or the like) will degrade. As such, when one of the detachable segments 206a to 206d is detached, the self-renewing end 202 will be renewed and the effectiveness of the laser treatment can be maintained at an optimal level.

In the discussion, unless otherwise stated, adjectives such as “substantially” and “about” modifying a condition or relationship characteristic of a feature or features of an embodiment of the disclosure, are understood to mean that the condition or characteristic is defined within tolerances that are acceptable for operation of the embodiment for an application for which it is intended. Unless otherwise indicated, the word “or” in the description and claims is the inclusive “or” rather than the exclusive or, and indicates at least one of, or any combination of items it conjoins.

While the presented concepts have been described with respect to a limited number of embodiments, it will be appreciated that many variations, modifications, and other applications of the disclosure can be implemented without departing from the scope of the appended claims.

What is claimed is:

1. A medical optical fiber, comprising:
a non-renewable portion; and
a renewable portion coupled to a distal end of the non-renewable portion, the renewable portion comprising a plurality of detachable segments.

2. The medical optical fiber of claim 1, wherein each of the plurality of detachable segments comprises an optically transparent material having a generally cylindrical shape.

3. The medical optical fiber of claim 1, wherein each of the plurality of detachable segments has a length between 0.2 millimeters (mm) and 6.0 mm.

4. The medical optical fiber of claim 1, each of the plurality of detachable segments comprising a ferrous material, the ferrous material configured to interact with an electromagnetic field to selectively decouple one of the plurality of detachable segments.

5. The medical optical fiber of claim 4, the medical optical fiber configured to be inserted through a working channel of an endoscope, the endoscope comprising a catheter having one or more electromagnets disposed at a distal end, the one or more electromagnets configured to generate the electromagnetic field.

6. The medical optical fiber of claim 1, wherein the plurality of detachable segments are coupled to each other via an adhesive material.

7. The medical optical fiber of claim 6, wherein the adhesive material is configured to melt, dissolve, or substantially weaken when exposed to specific wavelengths of light, when exposed to a select combination of laser pulse frequency and/or power, upon reaching a specific temperature, or upon exposure to a quantity of transmitted laser energy.

8. The medical optical fiber of claim 6, wherein the adhesive material between a first one of the plurality of detachable segments and a second one of the plurality of detachable segments is configured to melt when exposed to a first quantity of laser energy, wherein the adhesive material between the second one of the plurality of detachable segments and a third one of the plurality of detachable segments is configured to melt when exposed to a second quantity of laser energy, wherein the second one of the plurality of detachable segments is proximal to the first one of the plurality of detachable segments, wherein the third one of the plurality of detachable segments is proximal to the second one of the plurality of detachable segments, and wherein the second quantity of laser energy is higher than the first quantity of laser energy.

9. The medical optical fiber of claim 1, wherein each of the plurality of detachable segments comprises a machined end surface, wherein the machined end surface of a first one of the plurality of detachable segments is configured to attract via intermolecular attraction the machined end surface of a second one of the plurality of detachable segments when the end surfaces are positioned proximate to each other.

10. An endoscope system, comprising:
an endoscope configured for use with a renewable optical fiber, the endoscope having a working channel; and
a renewable optical fiber configured to be inserted through the working channel, the renewable optical fiber comprising:
a non-renewable portion; and
a renewable portion at a distal end of the non-renewable portion, the renewable portion comprising a plurality of detachable segments.

11. The endoscope system of claim 10, the endoscope comprising a catheter, the working channel extending from a proximal end to a distal end of the catheter, the distal end of the catheter comprising a cleaving apparatus configured to cleave the plurality of detachable segments.

12. The endoscope system of claim 11, the endoscope comprising a handle, the handle comprising an actuator arranged to actuate the cleaving apparatus.

13. The endoscope system of claim 10, wherein each of the plurality of detachable segments comprises an optically transparent material having a generally cylindrical shape.

14. The endoscope system of claim 10, wherein each of the plurality of detachable segments have a length between 0.2 millimeters (mm) and 6.0 mm.

15. The endoscope system of claim 10, the endoscope comprising a catheter, the working channel extending from a proximal end to a distal end of the catheter, the distal end of the catheter comprising one or more electromagnets, wherein each of the plurality of detachable segments comprise a ferrous material, the ferrous material configured to interact with an electromagnetic field generated by the one or more electromagnets to selectively decouple a one of the plurality of detachable segments.

16. A lithotripsy method comprising:
providing a medical optical fiber having a plurality of detachable segments coupled to a distal end of the medical optical fiber;
inserting the medical optical fiber through a working channel of an endoscope;
irradiating a target with laser energy via the medical optical fiber; and
renewing the distal end of the medical optical fiber by detaching a most distal one of the plurality of detachable segments.

17. The lithotripsy method of claim 16, wherein each of the plurality of detachable segments comprises an optically transparent material having a generally cylindrical shape.

18. The lithotripsy method of claim 16, wherein each of the plurality of detachable segments has a length between 0.2 millimeters (mm) and 6.0 mm.

19. The lithotripsy method of claim 16, each of the plurality of detachable segments comprising a ferrous material, the ferrous material configured to interact with an electromagnetic field to selectively decouple one of the plurality of detachable segments.

20. The lithotripsy method of claim 19, the endoscope comprising a catheter having one or more electromagnets disposed at a distal end, the one or more electromagnets configured to generate the electromagnetic field.
